(19) United States
(12) Patent Application Publication
(10) Pub. No.: US 2011/0281339 A1
(43) Pub. Date: Nov. 17, 2011

(54) System And Method To Create A Traveling Wave Within A Photobiotic Reactor To Enhance Algae Growth

(75) Inventors: LANCE W. RILEY, BROWNSVILLE, TX (US); BRAD W. BARTILSON, RANCHO VIEJO, TX (US); EVAN B. BARTILSON, RANCHO VIEJO, TX (US); DINESH KUMAR ARORA, MELBOURNE, FL (US); STEPHEN P. GREER, BROWNSVILLE, TX (US)

(73) Assignee: PHOTON8, INC., BROWNSVILLE, TX (US)

(21) Appl. No.: 12/780,617

(22) Filed: May 14, 2010

(51) Int. Cl. C12N 1/12; C12M 1/04

(52) U.S. Cl. 435/257.1; 435/292.1

(57) ABSTRACT

A photobioreactor with a gas input is disclosed herein. The photobioreactor is tilted about an axis of rotation at various angles to provide for various flow patterns of bubbles created by the gas input. The flow patterns may vary, but include: bubble flow; slug or plug flow; churn; annular flow; or wispy annular flow. In certain configurations, a bubble flowing through the photobioreactor may increase algae growth within the reactor.

### RELATED APPLICATIONS

This application is related in subject matter to PCT Patent Application No. PCT/US2010/029045 filed Mar. 29, 2010, and to U.S. patent application Ser. No. 12/414,149, filed Mar. 30, 2009, which are hereby incorporated by reference.

### TECHNICAL FIELD

This application relates generally to photobioreactors.

### BACKGROUND

Primary requisites for algal growth systems are photon acceptance, water, trace nutrients, and a carbon source. Carbon dioxide is a common choice for the carbon source as it is an environmentally-destructive gas (aka “greenhouse gas”) which can be extracted from the stack emissions of electrical generating facilities. With proper control of the requisite ingredients, algae can be grown and harvested continuously during sunlight hours.

There are two basic types of algal growth systems-open and closed systems. Open systems (aka “open ponds” or “open raceway” systems) consist of an enclosed pond in which the algae are fed nutrients, CO₂ and are directly exposed to sunlight to permit photosynthesis. In the open raceway configuration the pond is an oval shape with a central divider and paddle wheel to induce continuous flow around this oval “race track”. U.S. Pat. No. 1,643,273 teaches the basic concept of continuous loop raceway for aquaculture.

The Department of Energy demonstrated the production of biodiesel from algae in its “Aquatic Species Program” in operation from 1979-1996. This program, while forefronting algae biofuels production, found its process non-competitive with fossil fuels, with issues of species invasion (the directed algae were quickly overcome by indigenous algae species of a lower lipid content), evaporation, and high processing costs. Open ponds have direct exposure to all environmental events. Additionally, the fixed nature of open pond design prevents change for future design enhancements and/or reconfiguration for plant layout modification. The construction of such systems typically exceeds $100/m2. On a ten year basis, the amortized yearly cost of open ponds is $10/m2, even ignoring the time value of money. Operating costs have recently been reported as low as $30/m2, yet this still renders oil cost over $10/gallon. The economics render the systems commercially impractical.

Covers have recently been added to open raceway systems, e.g. US Patent Applications Nos. 20080178739 and 2008299643. This addition lessens the environmental effects, and can reduce evaporation and improve the thermal control of the system. The cover however adds to the cost basis. And the reduced sunlight delivered to the pond surface will further erode photosynthetic performance. Yusuf Christi in “Biodiesel from microalgae” research paper in Biotechnology Advances 25 (2007) reports findings of open ponds without covers exhibit 37% lower biomass and oil yield relative to closed systems or “photobioreactors”.

First generation closed systems or “photobioreactors” utilized transparent tubes made of rigid plastic (e.g. acrylic) through which the algal broth flows. The closed system provides isolation from environmental events and infiltration from other species. Greater process control is achieved, as evidenced by the higher productivity. This design is somewhat more available to design change and reconfiguration. US Patent 20090011492 teaches the use of large diameter acrylic tubes held at a highly inclined angle and having internal recirculation paths within the tubes.

While averting or reducing the drawbacks of open pond systems, the acrylic tube photobioreactors have been shown to be prohibitively expensive. Typical characteristic costs are approximately $190/m2, thus rendering this approach economically unsustainable. Further, research has shown that in dense broth processes (process efficiency is generally improved with higher broth density) light does not penetrate far into the broth within the tube, leaving a large dark zone.

Others have developed light-pipe systems to increase the volumetric efficiency of photobioreactors. McCall in patent applications 20080268302 and 20080220515 teaches the use of parallel, edge transmitting devices mounted within the cultivation zone, to increase the depth of the photosynthetic activity. Wilson in patent application 20080160591 describes transparent panels having extended, light transmissive surfaces attached to the light impinged surface thereby extending the depth of light penetration. An alternative approach, wherein the light is gathered in solar concentrating systems and then delivered by light emitting fibers into the algae broth is described by Ono and Cuello in Design Parameters of Solar Concentrating Systems for CO2 Mitigating Algal Photobioreactor” The University of Arizona, “Energy” 29: 1651-1657. Therein the light transfer efficiency is stated to now be improved to 45%.

More recently, transparent film has been used in photobioreactors to achieve lower cost. Kerz in patent application 20080274494 teaches the construction of vertically-held sheets of plastic joined in such manner as to create horizontal flow channels which cascade downward in serial fashion, top-to-bottom as driven by gravity. Constructed in this manner, significant surface area can be developed per unit of floor area. The sheets are suspended and mechanically-rotated within a greenhouse enclosure. While this approach leverages a lower cost photobioreactor material, the added costs of the machinery and the surrounding greenhouse greatly challenge profitable operation.

Alternatively, Sears in patent application 20070048848 teaches the use of large and long transparent bags configured in dual-arrangement, having CO2 injected into the algae broth at one end connecting the two bags, and water/nutrients and harvesting occurring at the opposite connection end. Motion is imparted to the broth via a weighted roller mechanical drive over the bag, thereby squeezing the broth down the bag, in peristaltic manner. The arrangement is then similar to an open-raceway system, yet being enclosed in the bag. Therein, an elaborate containment and track support structure is displayed, impacting the design flexibility and challenging the cost model.

Cloud, in patent application 20080311649 displays a parallel arrangement of 6 inch diameter tubes made of transparent film, The separate tubes are pressured by the pumped algae broth, with no internal means of interconnection along the pathway, nor a novel means of end connection to avert substantial fitting cost. The large size of the tube induces large, unproductive dark zones.

### SUMMARY

The presently disclosed subject matter is directed to methods and systems for providing a traveling gas wave in an algae-based photobioreactor. In a configuration, a gas input is installed on a photobioreactor reactor. The gas may vary according to the application, but may include, but is not limited to, air, carbon dioxide, nitrogen, or mixtures thereof. The reactor is tilted at an incline so that when a gas bubble is introduced into the reactor from the gas input, the gas bubble travels along the reactor from the lower end to the higher end of the reactor. In a configuration, the incline and the amount of gas input into the reactor is adjusted to create a specific flow pattern.

The flow patterns may vary, but may include: bubble flow, where the liquid suspending the algae is continuous with a dispersion of bubbles in the liquid; slug or plug flow where the bubbles of gas collect and form larger bubbles whose diameters are close to the diameter of the reactor; churn flow in which the bubbles have broken down, thus causing oscillating churn regime; annular flow in which the bubbles are of such size as to cause depression of the liquid onto the walls of the reactor; and wispy annular flow in which portions of the liquid are intermixed with the gas.

Without limiting the disclosed subject matter to any one theory of operation, it is contemplated that creating a flow pattern, especially plug or slug flow, creates beneficial conditions in an algae-based reactor. For example, the traveling bubble wave may resuspend algae that may have settled on the bottom side of the reactor. In another example, the bubble may create a depression in the liquid that causes a larger surface area of the algae suspended in the liquid to receive light for energy production. In another example, the traveling bubble wave may help to remove the oxygen produced by the algae, shifting the photosynthesis reaction equilibrium towards increased production of oxygen (by reducing the partial pressure of oxygen), thereby alleviating growth limitations imposed by oxygen enrichment.

In certain embodiments, a substantially linear reactor is provided, the reactor comprising a liquid having algae suspended or contained within the liquid (“algae broth”). The reactor is tilted about an axis so that one end of the reactor is higher than the other end, with the angle of tilt determined based upon operating conditions. The reactor further comprises a gas inlet that is configured to periodically or on an on-demand basis introduce a bubble of the gas into the end of the reactor that is lower than the other end. The gas is preferable introduced at the termination point of the lower end, but may be introduced along any point of the reactor.

In another embodiment, a method of algae growth is disclosed wherein an algae broth is dispersed within a hollow reactor having two ends. The reactor is tilted about an axis of rotation to provide for one end being elevated higher than the other. A gas inlet is configured to input a volume of gas into the lower end of the reactor, the amount configured to create a desired gas flow within the reactor. In some configurations, the amount of gas input causes bubble flow, slug or plug flow, churn flow, annular flow or wispy annular flow.

In one embodiment, a section of the reactor is slightly lifted, after the point of air injection, causing smaller injected bubbles to collect and form larger bubbles, which then progress upwardly, imparting a “slug” flow.

In another embodiment, CO2 or other carbon containing gas may be injected with the air to provide a carbon source for photosynthesis (all configurations applicable).

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Other features of the subject matter are described below.

### BRIEF DESCRIPTION OF THE DRAWINGS

The foregoing and other aspects of the present subject matter will become apparent from the following detailed description of the subject matter when considered in conjunction with the accompanying drawings. For the purpose of illustrating the subject matter, there is shown in the drawings embodiments that are presently preferred, it being understood, however, that the subject matter is not limited to the specific instrumentalities disclosed. The drawings are not necessarily drawn to scale. In the drawings:

FIG. 1 is an illustration of an exemplary and non-limiting parallel photobioreactor (“PFR”) in an unpressurized state;

FIG. 2 is an illustration of the exemplary and non-limiting PFR of FIG. 1 in the pressurized (working) state;

FIG. 3 illustrates the elliptical form of an exemplary and non-limiting PFR flow channels;

FIG. 4 is an illustration showing an exemplary and non-limiting PFR tilted about an axis with a gas input; and

FIG. 5 is a side view illustration showing plug flow through an exemplary and non-limiting PFR.

### DETAILED DESCRIPTION OF ILLUSTRATIVE EMBODIMENTS

The present subject matter may be understood more readily by reference to the following detailed description taken in connection with the accompanying figures and examples, which form a part of this disclosure. It is to be understood that this subject matter is not limited to the specific devices, methods, applications, conditions or parameters described and/or shown herein, and that the terminology used herein is for the purpose of describing particular embodiments by way of example only and is not intended to be limiting of the claimed subject matter.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Although the following description may be directed to one or more certain configurations of PFR, it should be understood that the present subject matter is not limited to any specific configuration and may be used in various reactors having various geometric shapes that can support or create the various flow patterns discussed above and below. Further, although the description may focus at times on plug or slug flow, it should be understood that the subject matter is not limited to any one specific flow pattern.

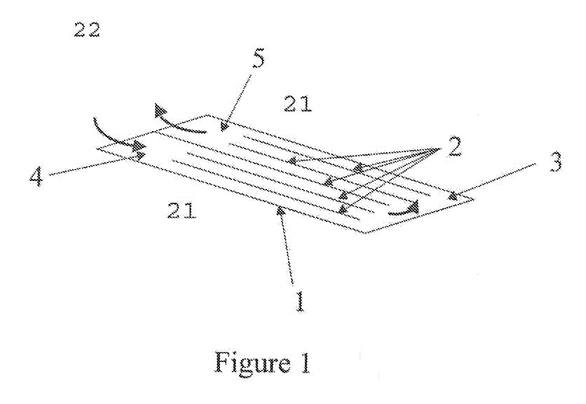

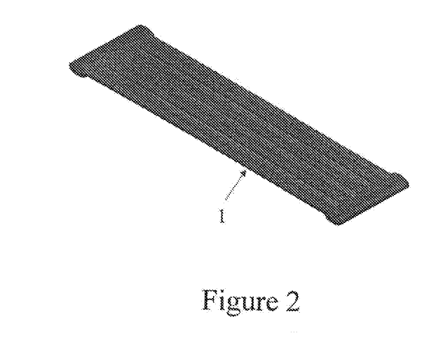

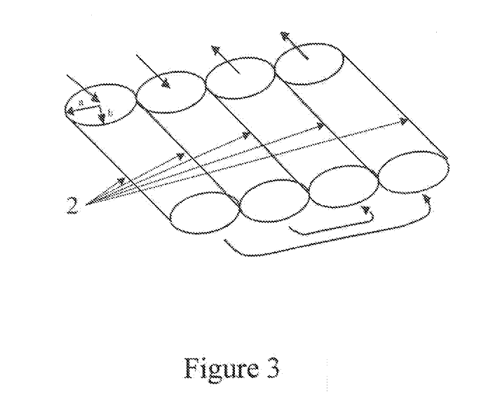

In the present exemplary and non-limiting PFR, an upper and lower sheet of film (upper layer being transparent) may be joined in such manner as to create flow channels between the sheets. The PFR of FIG. 1 is shown with seams 2 joining the sheets to form flow channels 21 therebetween, and a divider 22 between opposing flow sections of the PFR 1. The flow channels 21 may be combined at manifolds 4 and 5 where the flow enters and exits the PFR 1. The manifolds may also serve to return the flow, without the use of connectors to the same end of the PFR 1 as shown in FIG. 1. Once pressurized by the working fluid (algae “broth”) the flow channels and the manifolds become inflated to the working geometry as shown in FIG. 2. Due to the slight asymmetry of the joint geometry, the flow channels my take on a slightly elliptical shape as shown in FIG. 3.

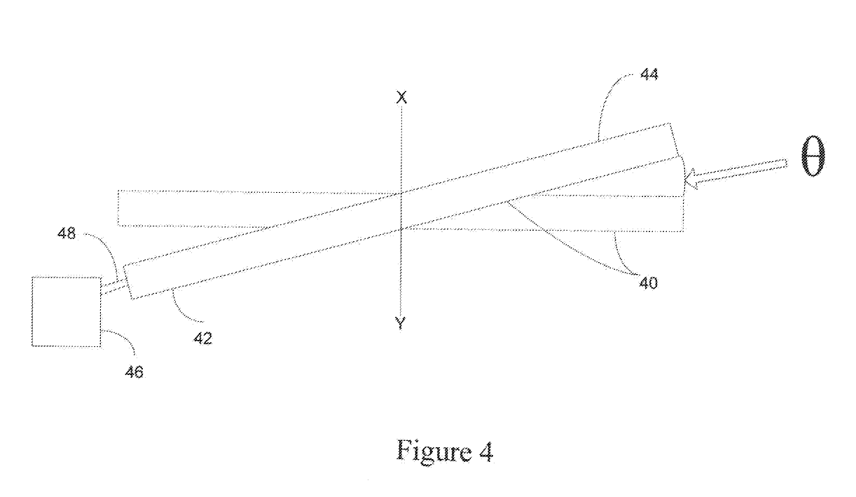

To preferably increase algae growth, an exemplary PFR is shown in FIG. 4, wherein the PFR is tilted. PFR 40 (illustrated by dotted lines) is tilted about axis XY at angle θ so that end 42 of PFR 40 is at a lower elevation than end 44 of PFR 40. Angle θ may be adjusted for various reasons including, but not limited to, providing for the desired gas flow configuration. As angle θ is increased while the geometric shape and size of PFR 40, as well as its contents, remain the same, the amount of gas added from gas device 46 via gas input 48 may also be adjusted to provide for desired internal conditions of PFR 40.

Further, the geometry of PFR 40 may be adjusted to provide for the ability to create certain types of flow patterns. For example, if the inner diameter of PFR 40 is significant (e.g. greater than 4″ in some configurations), the amount of gas necessary to create a plug flow may be beyond what the structural limitations of PFR 40 can withstand. Thus, while the presently disclosed subject matter is not limited to any specific inner diameter, the inventor has contemplated that the combination of placing the tubes of PFR 40 in an angle that is approximately 1 degree to approximate 30 degrees off of horizontal while adjusting the inner diameter to allow for flow conducive to algae growth, the operation and output of PFR 40 may be favorable adjusted.

For example, increasing θ while maintaining gas input constant may increase the velocity of the flow of a gas bubble through PFR 40 but may also change the flow pattern. In other words, a faster flow may change the flow pattern from slug flow to annular flow. In the same manner, increasing the gas input while maintaining θ constant may increase volumetric flow rate through PFR 40 but also change the flow pattern. Along with the geometry of PFR 40, as well as the contents of PFR 40, angle θ and gas input through gas inlet 48 affects the flow pattern.

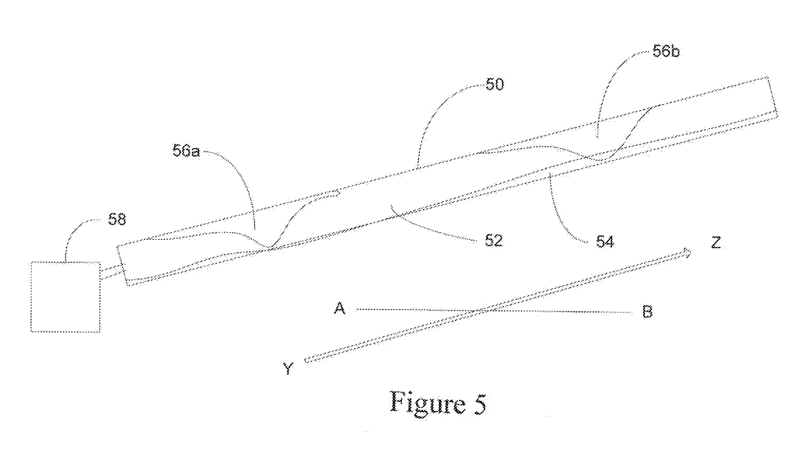

FIG. 5 illustrates an exemplary, tilted PFR. Gas input 58 inputs a certain amount of gas into PFR 50. PFR 50 has within its chambers an algae broth 52, a mixture of, amount other things, water and algae. The broth may also be comprised of additional materials such as plant food. Gas input 58 cause plugs 56a and 56b to form within PFR 50, which is substantially hollow. Plug 56a travels upward along line YZ from Y to Z. Line YZ is shown as having an angular displacement about horizontal line AB. Horizontal line AB is representative of a line that is parallel to the gravitational pull of the Earth. Plug 56b, further up PFR 50, was formed prior to plug 56a and shows how a plug flows through PFR 50.

FIG. 5 also illustrates how the plug is formed by the periodic or on demand input of gas via gas input 58. Gas input 58 may be configured to create a burst of gas to create plugs 56a and 56b, may be constant so that only once the volume of gas from gas input 58 is sufficient does plug 56a or 56b form and move, or may be configured in other ways to provide for a desired flow pattern at a desired frequency. As plug 56a or 56b travels up PFR 50, algae 54, which has settled on the bottom of PFR 50, may be mixed back into algae broth 52 via the mechanical action of plug 56a or 56b upon algae 54.

### EXPERIMENTAL DATA

The presently disclosed subject matter was tested to determine any change in growth of algae (dry weight of algae, per volume, per day or DW/cm2/day). The following are intended to provide additional information regarding various aspects of the presently disclosed subject matter and is not intended to limit the scope of the application to any of the following configurations. Air-life photobioreactors have been employed to pump algae without cell damage, however, compressing air for the hydraulic lifting purpose, particularly for a large production system is energy inefficient and cost prohibitive. In the invention of this application, a low shear pump was used to circulate the algae. In manner of this invention, the hydraulic lifting effort and the air effect is obtained separately. The slug flow effect is obtained with a air/gas injection volume rate that is very low (1 cubic foot/hour for a two-channel PFR). The slug flow effect is highly synergistic with the circulation effect, in that the stirring of the slug flow greatly reduces the demand on circulation (pumping) to stir (mix) the algal broth. In a specific configuration where broth flow is downhill, the gas is injected at the low end, hence the gas flow is opposite to the fluid flow, demonstrating the separation of the two requirements (fluid flow and slug/air flow for gas release) and yet providing high synergistic response.

### Experiment #1

An in-line degas/regas canister (e.g. a gas input) was installed on a PFR input line on a PRF. This canister is similar to low head degassing canisters used in commercial aquaculture operations, except modified without a gas outlet port. Upon installation, it was noticed that a void on input end of PFR stayed partially inflated and upon filling would periodically develop a bubble in the PFR channel. The bubble traveled to the output end of the PFR when on 1% grade. It appeared that this travelling bubble periodically resuspended any settled material. This “Traveling Wave” effect may be similar to a Taylor bubble effect in an air-lift system, in that the large bubble circumference (which encompasses or is substantially the same as the tube inner circumference) may be limited by tube diameter. In the present experiment, it was found that the current <2″ diameter in the PFR optimizes this effect. In this experiment, the PFR tubes were near horizontal and not vertical as in an air-lift, meaning that the shape of the bubble in the PFR is more like a V instead of a “pancake” shape as found in an air-lift.

In the present experiment, it was found that the use of the traveling wave 1) increased growth (0.52 g DW/m2/day without canister, 1.14 g DW/m2/day with the canister); 2) improved buoyancy (average 65% algae floating without the canister, >90% floating with the canister); 3) increased oxygen release (dissolved oxygen levels did not spike to >100% saturation with the canister compared to near 120% saturation without the canister); and 4) created a high maximum standing crop biomass (around 1.0 g DW/L with the canister to >0.7 g DW/L). This comparison was performed using similar inoculum, seawater, lighting and nutrient constituents and the same PFR in repetition. It should also be noted that >90% floating for short periods of time early in the growth cycle is common, however the degas/regas canister addition in the PFR supply line with the consequent “Traveling Wave” caused maintenance of >90% floatation through late growth phase.

### Experiment #2

In this experiment, ⅛″ tubing bulkhead fittings were added to the channel tubes of a 4′, 3-channel PFR 35 with an attached aerator and needle valves. Fittings were placed approximately 2″ into the channel. By elevating the input pillow only slightly, the same “Traveling Wave” could be attained without the use of the degas/regas canister. This unit was operated with a clean culture and production rate of 2.2 g DW/m2/day with the same improved buoyancy, oxygen release and maximum standing crop. No other strict comparisons with controlled conditions were made at the time since other PFRs were in operation from difference inoculum and seawater.

### Experiment #3

An extended study was performed to further test the traveling wave of the presently disclosed subject matter. The present experiment (consisting of multiple tests) were started with similar inoculum, seawater, light and nutrients. Data concerning PFR construction and operation are presented in the table below. One exception is PFR 35, which was started with good inoculum. PFRs started after April 16 were inoculated with regenerated old PFR 5 plates which we have since determined have had a significant impact on contamination and growth rate.

| | | | | | | Production | Previous |
|---|---|---|---|---|---|---|---|
| Date | | | | PFR | Bouyancy | Rate g | Production |
| Started | PFR # | # of Channels | Air Injection | Length | Observations | DW/m2/day | Rate |
| 4/16 | 26 | 3 | none | 4′ | <70% floating | 0.98 | 0.69 |
| 4/16 | 37/38 | twin 2-channel | in-channel, | 20′ | >90% floating | 7.46 | n/a |
| | | in parallel | concurrent | | | | |
| | | | return | | | | |
| 4/16 | 17 | 3 | degas/regas | 4′ | >90% floating | 1.46 | 1.0 w/ |
| | | | canister | | | | canister, |
| | | | | | | | 0.52 w/o |

From this initial data, we can infer that the “Traveling Wave” effect is aiding in increasing PFR performance overall and dissolved oxygen measurements indicate a degassing effect by the air injection. It also appears that the “Traveling Wave” effect is especially productive in longer PFRs and has increased production in PFRs with more channels, which was previously unobtainable, keeping in mind that these high productivities could be reached in 2-channel PFRs where we believe that the short PFR residence time may allow degassing of oxygen. By adding this air injection system to the PFRs it appears that longer lengths are quickly obtainable without sacrificing production. More analysis is of the air-injection system especially in longer PFRs and in outdoor situations is needed along with highly controlled experiments without variables introduced after start-up. Note all the experiments were performed in light-limited conditions (in-lab, fluorescent fixure providing 25 uE/m2-s) for comparison. Outdoor lighting will enable significantly higher photosynthetic growth (1200-2000 uE/m2-s). In some configurations, growth rate may increase up to and beyond 7.0 gm/M2/day.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment but rather should be construed in breadth and scope in accordance with the appended claims.

What is claimed:

1. A photobioreactor comprising:
a hollow structure tilted at an angle ranging from approximately 1 degree from horizontal to approximately 30 degrees from horizontal about an axis so that a first end of the structure is at a higher elevation than a second end of the structure;
an algae broth contained within the hollow structure; and
a gas inlet configured to input a volume of gas into the hollow structure.

2. The photobioreactor of claim 1, wherein the hollow structure is comprised of:
an upper layer of transparent film and a lower layer of film wherein the upper layer and lower layer are attached to each other along the perimeter to form a sealed structure, the upper layer and lower layer also attached to form pathways comprising independent channels within the confines of the outer perimeter; and
a first manifold and second manifold on opposite ends of the parallel channels within the sealed structure wherein a first portion of the first manifold is in fluid communication with each of an inlet of a first subset of the channels and a second portion of the first manifold is in fluid communication with the first end and the second end and wherein the second manifold is in fluid communication with the first end and the second end.

3. The photobioreactor of claim 1, wherein the volume of gas is configured to provide for a flow pattern of a gas bubble comprising bubble flow; slug or plug flow; churn; annular flow; or wispy annular flow.

4. The photobioreactor of claim 3, wherein the flow pattern is slug or plug flow.

5. The photobioreactor of claim 3, wherein the circumference of the gas bubble is substantially the same as the inner circumference of the hollow structure.

6. The photobioreactor of claim 1, wherein the gas is carbon dioxide, nitrogen, air, oxygen, air, or mixtures thereof.

7. A method of algae production comprising:
providing a photobioreactor having a hollow structure;
inserting an algae broth within the hollow structure;
tilting at least a portion of the hollow structure about an axis of rotation so that a first end of the hollow structure is at a higher elevation than a second end of the hollow structure; and
inputting a volume of gas into the hollow structure to generate a slug or plug flow pattern.

8. The method of claim 8, wherein tilting at least a portion of the hollow structure comprising tilting at least a portion of the hollow structure at an angle ranging from approximately 1 degree from horizontal to 30 degrees from horizontal.

9. The method of claim 7, wherein the circumference of the gas bubble is substantially the same as the inner circumference of at least a portion of the hollow structure.

10. The method of claim 7, wherein the gas is carbon dioxide, nitrogen, air, oxygen, air, or mixtures thereof.
